(19) United States
(12) Patent Application Publication
(10) Pub. No.: US 2002/0142322 A1
(43) Pub. Date: Oct. 3, 2002

(54) Method for evaluating the intensity of muscular proteolysis

(75) Inventors: Marc Ferrara, Beaumont (FR); Christiane Deval, Chamalieres (FR); Daniel Bechet, Manzat (FR)

Correspondence Address: Mark B. Wilson, Fulbright & Jaworski L.L.P., Suite 2400, 600 Congress Avenue, Austin, TX, 78701, US

(73) Assignee: Institut National de la Recherche Agronomique: INRA, Paris (FR)

(21) Appl. No.: 09/943,272

(22) Filed: Aug. 30, 2001

Related U.S. Application Data
(63) Continuation of application No. PCT/FR00/00512, filed on Mar. 1, 2000.

(30) Foreign Application Priority Data
Mar. 2, 1999 (FR) 99 02582

(51) Int. Cl. C12Q 1/68; G01N 33/53

(52) U.S. Cl. 435/6; 435/7.1

(57) ABSTRACT

The invention relates to a method for evaluating muscular proteolysis. The invention is essentially characterized in that it consists in determinining the Cathepsin L content and/or mRNA content thereof in a muscular sample or blood sample from the human or animal body and includes a muscular proteolysis evaluation agent in order to carry out said method.

The subject of the present invention is a method for evaluating the intensity of muscular proteolysis and an agent for evaluating the intensity of muscular proteolysis.

In humans and mammals, the importance of the processes of intracellular proteolysis appears in the multiplicity of the sectors where it is involved. Through protein renewal, the proteolytic activities determine the accretion of proteins, thus determining, in the same way as the protein synthesis, the intensity of growth. These activities also occur in the phenomena of cell proliferation and transformation and in several conditions. Certain stress situations (sepsis, multiple traumas, extensive burns, AIDS, presence of tumors and the like) are accompanied by muscular protein fusion, a first visible manifestation of an activation of all the intracellular proteolytic systems. Losses of urinary nitrogen of the order of 10 to 25 g/d in trauma patients can be doubled in the event of infection, which corresponds to the daily loss of 300 to 600 g of muscle.

It has therefore appeared appropriate to be able to evaluate muscular proteolysis at an early stage, before observing substantial muscular loss.

Furthermore, for practical reasons, a method for evaluating muscular proteolysis will need to be easy to carry out and to make it possible to obtain results rapidly. These results will need to be quantitative and/or qualitative.

After long research studies which will be described hereinafter, the applicant has developed a novel method which makes it possible to evaluate muscular proteolysis, fulfilling the practical conditions set out above, as well as an agent for evaluating the proteolysis which makes it possible to carry out this novel method.

In a first instance, the applicant has sought to establish a record of the genes whose level of expression was appreciably modified during an induction of muscular proteolysis.

Among the models of induction of muscular proteolysis, the single injection of live bacteria, E. coli, in rats, makes it possible to maintain these animals in a catabolic situation for several days and thus reproduces the closest metabolic modifications to those observed in humans. This catabolic situation is called “sepsis” in the remainder of the text.

To detect the genes which are overexpressed during muscular proteolysis, the applicant used the so-called “DD-RT-PCR” method (Differential Display-Reverse Transcription-Polymerase Chain Reaction). The principle of this technique is based on the combinatory amplification of DNA fragments from all of the messenger RNAs, called hereinafter mRNA, present in the muscular tissue of rats of the model described above, that is to say rats in a situation of sepsis, and control rats, which are not in a situation of proteolysis. The mRNAs isolated from these tissues are subjected to the action of reverse transcriptase in the presence of a primer anchored in 3′ on the poly A tail of the mRNAs. Next, an amplification step is carried out by PCR in the presence of a decamer playing the role of a primer in 5′. The amplification of the fragments representing all of the messengers is obtained by the successive use of several decamer-3′ primer combinations.

The amplification step is then carried out in the presence of a ³³P-labeled DNTP. The DNA fragments thus obtained are separated according to their size by polyacrylamide gel electrophoresis. The gels are then visualized by conventional autoradiography. The bands of the gels obtained using the DNA of rats in a situation of sepsis are compared with the bands of the gels obtained using the DNA of the control rats. The differential fragments, that is to say the bands present solely on the gel obtained using the DNA of rats in a situation of sepsis are then removed, reamplified, cloned and then sequenced. This sequence is then compared with all the nucleotide sequences present in DNA banks. Thus, the differential fragment may be identical to a known DNA sequence. By proceeding in this manner, the applicant has surprisingly identified a differential fragment.

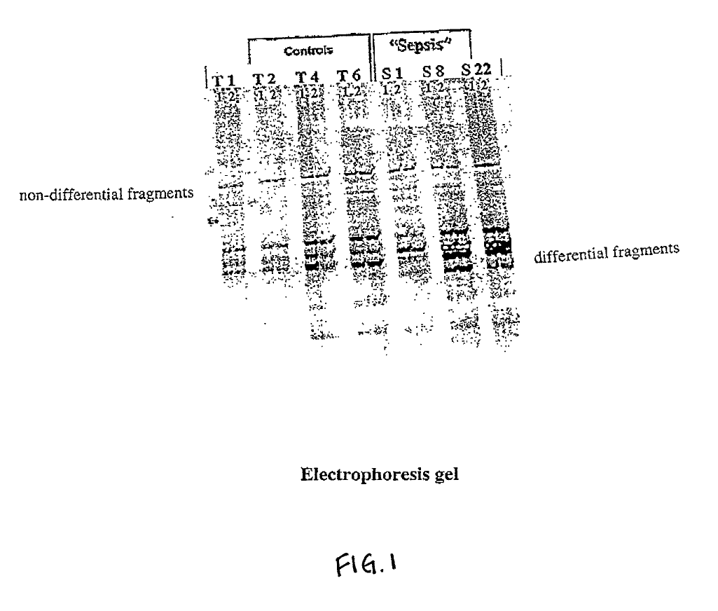

FIG. 1 represents the gel obtained after electrophoresis of DNA of control rats and of rats in a situation of sepsis; a differential fragment present solely in the samples of DNA of rats in a situation of sepsis is visible.

This fragment of 256 nucleotides, after having been sequenced, was compared to 325,789 sequences present in a DNA databank.

Unexpectedly, it was observed that it corresponds to the messenger encoding cathepsin L. The latter is a cysteine proteinase of the lysosome.

The differential character of this fragment was confirmed by analysis by protection from RNases and by Northern-blot analysis.

These experiments have made it possible to demonstrate that the mRNA content of cathepsin L was between 15 and 20 times higher in rats in a situation of “sepsis”, compared with the cathepsin L MRNA content in control rats.

In order to ensure that this increase in the level of cathepsin L mRNA observed in rats in a situation of sepsis is not a response specific to the bacterial infection, the applicant determined the cathepsin L mRNA content in the muscle in a proteolytic situation induced by causes other than bacterial infection.

It was observed that in rats carrying the Yoshida sarcoma (tumor), the cathepsin L mRNA content is greatly increased. During treatment with muscular proteolysis inhibitors such as pentoxyfilline and Hw 448, it was observed that the cathepsin L mRNA content decreases considerably.

A substantial increase in the cathepsin L mRNA content of muscles obtained from rats treated with dexamethasone, a hormone inducing muscular proteolysis, compared with untreated control rats, was also observed.

It was also observed that the difference in the cathepsin L mRNA content is specific to the muscular tissue since it was observed in two types of muscles collected, Gastrocnemius and Tibialis, whereas it is absent in the liver of the animals from which the muscles were removed.

The applicant has also studied the correlation between the mRNA level and the quantity of protein present in the muscle.

In the context of the present invention, the expression cathepsin L should be understood to mean the zymogenic form, also called M.E.P. (Major Excreted Protein) and the active form of this protein.

To do this, the applicant has carried out a Western-blot analysis which has made it possible to observe that the content of the zymogenic and active form of cathepsin L increased in rats in a situation of “sepsis” in proportions of 2 to 5 fold compared with their content in uninfected control rats.

The applicant, on the basis of these variations in the content of cathepsin L and/or of its mRNA, has thus isolated, unexpectedly, an indicator of the intensity of muscular proteolysis.

The present invention therefore relates to a method for evaluating muscular proteolysis, essentially characterized in that it consists in determining the content of cathepsin L and/or of its mRNA in a muscular or blood sample from the human or animal body.

Any analyses conventionally used which make it possible to determine the content of a protein qualitatively and/or quantitatively may be used in the context of the present invention.

The cathepsin L content may be determined, for example, using an antibody directed against all or part of the structure of cathepsin L, in the zymogenic or active form of this protein. This antibody may itself be labeled, that is to say, for example, coupled to a marker enzyme (direct immunological analysis). The anti-cathepsin L antibody/cathepsin L pair may also be visualized by a second labeled antibody directed against the anti-cathepsin L antibody (indirect immunological analysis).

Analyses using such antibodies are, for example, those known to persons skilled in the art called “Western blotting”, “ELISA” or “Histo-Immuno”.

The semiquantitative Western blot technique essentially consists in running the proteins of a muscular or blood sample on a gel, in transferring them onto a nylon membrane and then in placing the gel in the presence of anti-cathepsin L monoclonal antibodies, in visualizing these antibody-cathepsin L pairs using labeled antibodies, and finally in reading the gel thus obtained.

The ELISA technique, which is more appropriate for a quantitative determination, consists, in a first instance, in establishing a calibration series using known concentrations of cathepsin L and defining the corresponding optical values, and then in measuring the optical value for the sample and in accurately determining, using a calibration series, the cathepsin L concentration.

The Histo-Immuno technique, which is more appropriate for a qualitative determination, consists in carrying out a direct or indirect immunological analysis on the muscular tissue.

By comparing the intensity of the labeling of cathepsin L with the intensity of the labeling of a reference protein, the cathepsin L content and thus the presence of proteolysis can also be determined.

For example, if the concentration of the reference protein is relatively close to that of cathepsin L, and if its concentration does not vary as a function of the proteolysis, two relatively similar labelings will be observed in the absence of proteolysis, whereas a much more pronounced labeling, corresponding to cathepsin L, will be observed in the presence of proteolysis.

Such reference proteins are in particular “housekeeping” proteins participating in the basal cell metabolism. Reference proteins may also be chosen from the proteins of the contractile structure. These proteins of the contractile structure are in particular actin and the myosins.

A reference protein may also be a protein whose content decreases during proteolysis, thus making it possible to obtain an even higher amplitude of variation. An example of such a protein is GAPDH.

In the context of the present invention, any technique conventionally used for a qualitative or quantitative determination of mRNA may be used.

The cathepsin L MRNA content may in particular be determined by hybridizing the mRNA of the muscular or blood sample from the human or animal body with a labeled probe complementary to this mRNA.

Such techniques are, for example, the techniques called Dot-blot, Slot-blot, Northern-blot, RPA, RT-PCR kinetics, competitive RT-PCR, mini- and micro-Array, and Chips. These techniques are known to persons skilled in the art.

All these techniques require the use of a probe specific for the cathepsin L MRNA. This probe may be a segment or any sequence complementary to the cathepsin L MRNA.

These probes may also be specific to the species for which the proteolysis is evaluated.

In humans, the preferred probe is the probe of sequence No. 1 corresponding to the oligonucleotide starting at the 141st nucleotide and ending at the 1161st nucleotide of the sequence of cDNA of the gene for human cathepsin L.

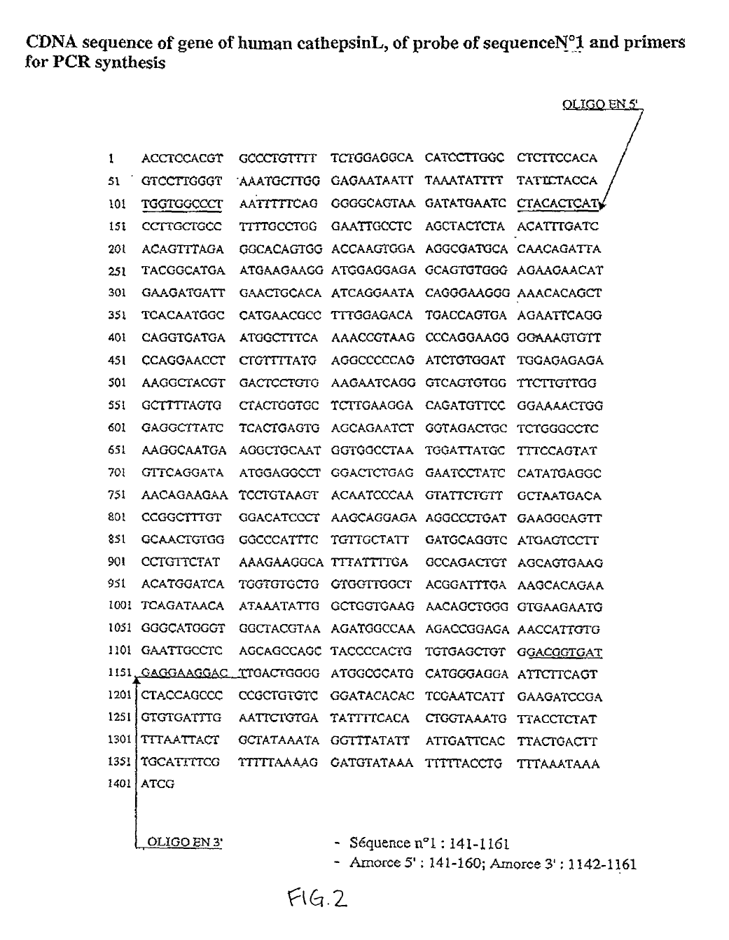

The subject of FIG. 2 is the CDNA sequence of the gene for human cathepsin L. Also represented on this sequence are the probe of sequence No. 1 and the primers in 5′ and 3′ which are used to amplify this sequence No. 1 by PCR.

For a qualitative analysis, the so-called Northern blot technique is preferably used.

For a quantitative analysis, the use of the technique called competitive RT-PCR is preferred.

Another object of the present invention consists in determining the cathepsin L MRNA content and, in addition, in determining the content of mRNA for a reference protein whose MRNA content is independent of the proteolysis or whose MRNA content decreases with proteolysis, and then in comparing the results obtained. Comparison of the two signals reflecting the MRNA content will thus make it possible to comparatively evaluate the proteolysis.

In the context of the present invention, the muscular proteolysis is preferably evaluated by comparing the cathepsin L messenger RNA content with the content of MRNA for a reference protein.

The subject of the present invention is also an agent for evaluating muscular proteolysis, essentially characterized in that it comprises at least one means which makes it possible to determine the content of cathepsin L and/or of its mRNA in a muscular or blood sample from the human or animal body.

This means allows the use of the methods described above.

Thus, the means which makes it possible to determine the cathepsin L content may in particular comprise antibodies specifically directed against all or part of the structure of cathepsin L. These antibodies may be labeled in the case of direct immunological analyses or the means may comprise other labeled antibodies anti(anti-cathepsin L antibodies) in the case of indirect immunological analyses.

The means making it possible to determine the cathepsin L mRNA content comprises, inter alia, a probe complementary to said mRNA. This means may also comprise a solid support, such as a nylon membrane, a glass or silicon plate, on which the probe complementary to the cathepsin L mRNA is attached.

For the evaluation of the proteolysis in humans, this means may in particular comprise the probe described in FIG. 2.

This means may also comprise compounds necessary for the labeling of the RNAs to be tested. The markers preferably used are not radioactive and may be read by fluorescence.

Another subject of the present invention consists in an agent for evaluating muscular proteolysis containing a first means which makes it possible to determine the content of cathepsin L and/or of its MRNA, and, in addition, a second means which makes it possible to determine the content of a reference protein and/or of its mRNA present in the sample and whose content is independent of the proteolysis or whose content decreases with the proteolysis.

These means may be for example solid supports such as those described above, on which the sequence complementary to the MRNA is attached.

The following examples are intended to illustrate the present invention without, as a result, exhibiting a limiting character.

### EXAMPLE 1

### Evaluation of the Intensity of the Muscular Proteolysis by Determining the Quantity of Cathepsin L Protein

The content of cathepsin L, zymogenic (“Pro”) and active (“Active”) form, was determined by a Western-blot analysis.

The Gastrocnemius muscles of control rats (PF) and rats infected (S) by an injection of E. coli were collected 2 and 6 days after infection. 200 mg of muscle are homogenized in a 20 mM phosphate buffer pH 6.25 containing 0.1% Triton. The homogenate is centrifuged at 15,000 g for 15 min at 4° C. The supernatant is diluted with an identical volume of SDS-PAGE buffer (Laemmly). Samples (20 μg of total proteins) are subjected to electrophoresis in the presence of SDS on a 12.5% polyacrylamide gel. The separated proteins are electrotransferred onto a polyvinylidene difluoride membrane in a 48 mM Tris-30 mM Glycine buffer pH 8.3 containing 20% (V/V) of methanol. The blots are saturated for 1 h with a phosphate buffer containing 0.2% (W/V) of skimmed milk and 0.1% of Triton X-100. The cathepsin L is visualized by chemiluminescence using the Aurora kit (ICN). The primary polyclonal antibody used is a rabbit IgG specific for cathepsin L (gift from Mr M. GOTTESMAN, Bethesda, USA). The secondary antibody is an anti-rabbit IgG obtained from goats and conjugated with alkaline phosphatase. The signals are visualized by STORM 484 and analyzed with the ImageQuant software (Molecular Dynamics).

The results obtained are the following:

The values are expressed in Molecular Dynamics Arbitrary Units.

Mean (standard deviation)−n=3

| | Cath. L “Pro” form | Cath L. “Active” form |
|---|---|---|
| | (Mol Dyn AU) | (Mol Dyn AU) |
| PF | 473 | 700 |
| S | 1243 | 3170 |
| Ratio | 2.6 | 4.53 |
| means S/PF | | |

Thus, it was observed that the content of cathepsin L, prozymogenic or active form, corresponds to 2 to 4.5 times the content of cathepsin L in the muscles of control animals.

### EXAMPLE 2

### Evaluation of the Intensity of Muscular Proteolysis by Determining the Cathepsin L mRNA Content

The Gastrocnemius and Tibialis muscles of control rats (T) and rats infected (S) by injection of E. coli were collected 6 days after infection.

The total RNAs of these Gastrocnemius and Tibialis muscles are extracted according to the Chomczynski and Sacchi technique (1987. Anal Biochem. 162, 156-159: Single step method of RNA isolation by guanidium thiocyanate-phenol-chloroform extraction).

The cathepsin L mRNA content is then determined by Northern-blot analysis.

After migration under denaturing conditions on a 1% agarose gel, 10 to 30 μg of RNAs are transferred onto nylon membranes (Hybond N⁺-Amersham) . The probes used in hybridization are labeled with the “ready to go DNA labeling” kit (Pharmacia Biotech) according to the manufacturer's instructions. The hybridizations are carried out at 65° C. for 16 to 20 h in the “Rapid-Hyb Buffer” (Amersham Life Science). The membranes are successively washed with the buffers 2X SSC-0.1% SDS, 0.5X SSC-0.1% SDS and 0.1X SSC-0.1% SDS for 20 min at 65° C. They are then placed on a “Phosphor Screen” and the signals are visualized and quantified using the PhospoFluroImager STORM 484 (Molecular Dynamics).

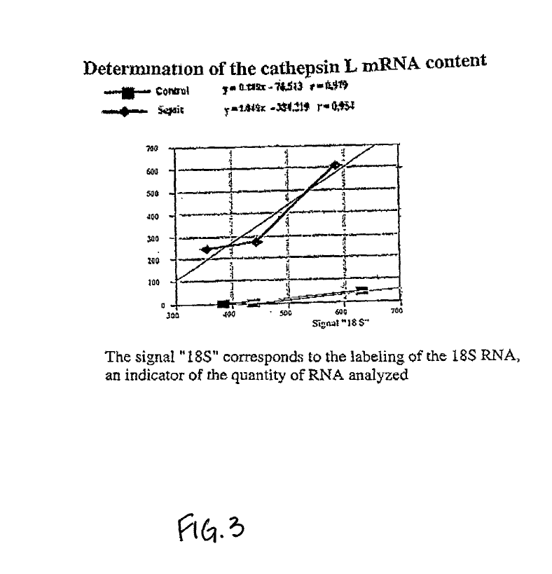

The results obtained are presented in FIG. 3.

The ratio of the slopes of the 2 straight lines in this figure represents the ratio of the cathepsin L mRNA contents.

It is observed that there is 8.7 times (1.649/0.189) more cathepsin L messenger in the muscle of the infected animal (S) than in that of the control animal (T).

### EXAMPLE 3

### Evaluation of the Intensity of Muscular Proteolysis by Determining the Relative Variaton of a Cathepsin L Messenger RNA Compared with that of a Reference Protein

The reference protein used is GAPDH. It was observed that the GADPH mRNA content decreases slightly during muscular proteolysis. Gastrocnemius (Ga.) and Tibialis (Ti.) muscles of control rats (T) and of rats infected (S) by injection of E. coli are collected 2 (D2) and 6 days (D6) after the infection.

50 ng of DNA of each of the targets, namely cathepsin L and GAPDH, are dotted on a nylon membrane Hybond N⁺ (Amersham). 5 to 25 μg of total RNAs extracted from the muscles of control and infected animals are denatured by treatment at 70° C. for 10 min in a volume of 14 μl in the presence of 2 μl of oligonucleotide dT25. The synthesis of the first strands complementary to the messenger RNAs is carried out using the SuperScript II kit (BRL) in the following reaction mixture:

| | |
|---|---|
| RNAsin 40 u | 1 μl |
| Buffer 5X | 6 μl |
| DTT 0.1 M | 2 μl |
| dATP, dGTP, dTTP, 20 mM | 1 μl |
| dCTP 120 μM | 1 μl |
| dCTP 32P 50 μCi | 5 μl |
| Enzyme SuperScript | 1 μl |

After 1 hour at 42° C., 1 μl of SuperScript is added to the mixture and the reaction is continued for 1 h at the same temperature. At the end of the reaction, the RNAs are degraded by a treatment with sodium hydroxide and the cDNAs synthesized are purified on a Sephadex G50 column.

The membranes are then prehybridized for 24 h at 68° C. and then hybridized at the same temperature for 60 h. After washes and exposure of the membranes on PhoporScreen, the signals are visualized (STORM Molecular Dynamics) and quantified using the ImageQuant software (Molecular Dynamics). The ratio of the mRNAs for cathepsin L and for GADPH is then calculated.

The following results were obtained:

| | D2 | | | | D6 | | | |
|---|---|---|---|---|---|---|---|---|
| | Ti. T | Ti. S | Ga. T | Ga. S | Ti. T | Ti. S | Ga. T | Ga. S |
| $\begin{matrix}Ratio \\ \frac{Cathepsin L mRNA}{GADPH mRNA}\end{matrix}$ | 0.01 | 0.12 | 0.02 | 0.18 | 0.05 | 1.74 | 0.07 | 0.45 |

The amplitudes of variation observed are very high.

Thus, the mRNA cathepsin L/GAPDH ratio makes it possible to evaluate muscular proteolysis with certainty.

1. A method for evaluating muscular proteolysis, characterized in that it consists in determining the content of cathepsin L and/or of its mRNA in a muscular or blood sample from the human or animal body.

2. The method as claimed in claim 1, characterized in that the cathepsin L content is determined using an antibody directed against all or part of the structure of cathepsin L.

3. The method as claimed in claim 1, characterized in that the cathepsin L mRNA content is determined by hybridizing said mRNA with a labeled probe complementary to said MRNA.

4. The method as claimed in claim 1 or 3, characterized in that the probe complementary to said mRNA corresponds to the probe of sequence No. 1.

5. The method as claimed in any one of claims 1 to 4, characterized in that it consists, in addition, in determining the content of a reference protein and/or of its mRNA present in the sample and whose respective content is independent of the proteolysis and whose content decreases with the proteolysis, and then in comparing the contents thus determined.

6. An agent for evaluating muscular proteolysis, characterized in that it comprises at least one means which makes it possible to determine the content of cathepsin L and/or of its mRNA in a muscular or blood sample from the human or animal body.

7. The agent for evaluating muscular proteolysis as claimed in claim 6, characterized in that the means which makes it possible to determine the cathepsin L content comprises antibodies directed against all or part of the structure of cathepsin L.

8. The agent for evaluating muscular proteolysis as claimed in claim 6, characterized in that the means which makes it possible to determine the cathepsin L mRNA content comprises a labeled probe complementary to said mRNA.

9. An agent for evaluating muscular proteolysis, characterized in that the probe complementary to said mRNA corresponds to the probe of sequence No. 1.

10. The agent for evaluating muscular proteolysis as claimed in any one of claims 5 to 9, characterized in that it contains, in addition, a second means which makes it possible to determine the content of a reference protein and/or of its mRNA present in the sample and whose respective content is independent of the proteolysis or whose content decreases with the proteolysis.
